(19) United States
(12) Patent Application Publication
(10) Pub. No.: US 2015/0245611 A1
(43) Pub. Date: Sep. 3, 2015

(54) ACTIVE INGREDIENT FOR CONTROLLING TRUE SPIDER MITES

(71) Applicant: BAYER CROPSCIENCE AG, Monheim (DE)

(72) Inventors: Wolfgang THIELERT, Odenthal (DE); Holger WECKWERT, Leichlingen (DE); Leonardo PITTA, Leverkusen (DE); Michael MAUE, Langenfeld (DE); Heiko RIECK, Burscheid (DE)

(21) Appl. No.: 14/635,271

(22) Filed: Mar. 2, 2015

(30) Foreign Application Priority Data
Mar. 3, 2014 (EP) 14157444.2

(51) Int. Cl. A01N 43/56; C07D 231/14

(52) U.S. Cl. CPC A01N 43/56; C07D 231/14

(57) ABSTRACT

The present invention relates to novel uses of the compound of the formula (I) for controlling pests/spider mites from the order of Acari.

### CROSS REFERENCE TO RELATED APPLICATIONS

This application claims priority from EP 14157444.2 filed Mar. 3, 2014, the content of which is incorporated herein by reference in its entirety.

### BACKGROUND

1. Field of the Invention

The present application relates to the use of a known active ingredient for controlling spider mites from the order of Acari in particular crops.

2. Description of Related Art

The compound of the formula (I)

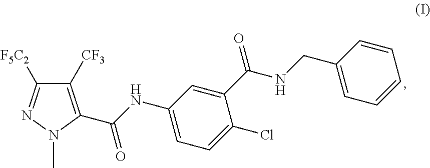

the preparation thereof and structurally similar compounds are known from WO 2010/051926. Further uses of this compound are also disclosed in WO 2010/051926.

Since the control of true spider mites in crops, especially field crops, is becoming increasingly important and the number of suitable active ingredients is falling, particularly because of regulatory demands in respect of ecotoxicological properties, the demand for suitable active ingredients is particularly high.

### SUMMARY

It has now been found that, surprisingly, the compound of the formula (I) is of particularly good suitability for controlling spider mites (Tetranychidae), especially for controlling spider mites from the species Tetranychus urticae, Tetranychus pacificus, Oligonychus pratensis, Oligonychus coffeae and Panonychus ulmi, in crops selected from green beans (Phaseolus vulgaris), soya beans (Glycine max), cotton (Gossypium), maize (Zea mays), tea (Camellia sinensis), coffee (Coffea, preferably Coffea arabica and Coffea canephora), grapes (Vitis vinifera, preferably Vitis vinifera L. subsp. vinifera), pome fruit, Solanaceae, stone fruit, preferably almonds (Prunus dulcis), Cucurbitaceae, and nut fruits, under the condition that the use of the compound of the formula (I) for controlling Tetranychus urticae in green beans (Phaseolus vulgaris) is excluded. In addition, the compound of the formula (I) also has favourable ecotoxicological properties.

The aspect of the present invention relates to the use of the compound of the formula (I)

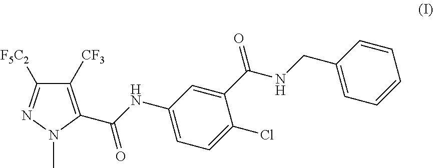

for controlling pests/spider mites from the order of Acari in crops selected from green beans (Phaseolus vulgaris), soya beans (Glycine max), cotton (Gossypium), maize (Zea mays), tea (Camellia sinensis), coffee (Coffea, preferably Coffea arabica and Coffea canephora), grapes (Vitis vinifera, preferably Vitis vinifera L. subsp. vinifera), pome fruit, Solanaceae, stone fruit, preferably almonds (Prunus dulcis), Cucurbitaceae, and nut fruits, under the condition that the use of the compound of the formula (I) for controlling Tetranychus urticae in green beans (Phaseolus vulgaris) is excluded.

### DETAILED DESCRIPTION OF A PREFERRED EMBODIMENT

One embodiment relates to the above use, characterized in that the spider mite is selected from Tetranychus urticae, Tetranychus pacificus, Oligonychus pratensis, Oligonychus coffeae, Polyphagotarsonemus latus and Panonychus ulmi.

A further embodiment relates to the above use, characterized in that the spider mite is a spider mite from the genus of Oligonychus. In one embodiment, the spider mite from the genus of Oligonychus is the spider mite Oligonychus pratensis. More particularly, it is possible to use the compound of the formula (I) for controlling Oligonychus pratensis in maize. In a further embodiment, the spider mite from the genus of Oligonychus is the spider mite Oligonychus coffeae. More particularly, it is possible to use the compound of the formula (I) for controlling Oligonychus coffeae in tea and coffee.

A further embodiment relates to the above use, characterized in that the spider mite is a spider mite from the genus of Tetranychus. In one embodiment, the spider mite from the genus of Tetranychus is the spider mite Tetranychus pacificus. More particularly, it is possible to use the compound of the formula (I) for controlling Tetranychus pacificus in stone fruit, more preferably in almonds, or grapes. In a further embodiment, the spider mite from the genus of Tetranychus is the spider mite Tetranychus urticae. More particularly, it is possible to use the compound of the formula (I) for controlling Tetranychus urticae in soya beans, cotton or maize.

A further embodiment relates to the above use, characterized in that the spider mite is Panonychus ulmi. More particularly, the compound of the formula (I) can be used for controlling Panonychus ulmi in a crop selected from the group consisting of cotton (Gossypium hirsutum), coffee (Coffea spec.), peanuts (Arachis hypogea), grapes (Vitis vinifera, preferably Vitis vinifera subsp. vinifera), and pome fruit (preferably apples (Malus domestica) or pears (Pyrus communis)), more preferably in a crop selected from the group consisting of grapes (Vitis vinifera, preferably Vitis vinifera L. subsp. vinifera) and pome fruit.

A further embodiment relates to the above use, characterized in that the spider mite is Polyphagotarsonemus latus. More particularly, the compound of the formula (I) can be used for controlling Polyphagotarsonemus latus in a crop selected from the group consisting of green beans (Phaseolus vulgaris), cotton (Gossypium hirsutum), tea (preferably Camellia sinensis and Camellia japonica), coffee (Coffea arabica), soya (Glycine max), peaches (Persea americana), blackberries (Rubus fruticosus) and raspberries (Rubus idaeus), grapes (Vitis vinifera, preferably Vitis vinifera L. subsp. vinifera), Solanaceae (preferably tomatoes (S. lycospersicum), bell pepper species (Capsicum genus), aubergines (Solanum melongena)), pome fruit (preferably apples and pears) and Cucurbitaceae, preferably cucumbers (Cucumis sativus), more preferably in a crop selected from the group consisting of green beans (Phaseolus vulgaris), cotton and Solanaceae (preferably tomatoes (S. lycospersicum), bell pepper species (Capsicum genus), aubergines (Solanum melongena)).

A further embodiment relates to the above uses, characterized in that the compound of the formula (I) is present in the mixture with at least one further insecticidal or acaricidal or nematicidal active ingredient.

A further embodiment relates to the above uses, characterized in that the compound of the formula (I) is present in the mixture with at least one further fungicidal active ingredient.

### DEFINITIONS

“Pome fruit” is a collective term both for fruits from flowering plants in the Malinae subtribe of the Rosaceae family and for the plants that form these fruits. Pome fruits include especially apples (Malus), cotoneasters, hawthorns (Crataegus), loquats (Japanese medlar; Eriobotrya), medlar (Mespilus), pears (Pyrus), firethorn (Pyracantha), toyon (Heteromeles), rowan (Sorbus) and quince (Cydonia). Particular preference is given to apples and pears.

Solanaceae are a family of angiosperms (Magnoliopsida). Solanum is the largest genus within the family. The Solanaceae include especially tomatoes (S. lycospersicum), bell pepper species (Capsicum genus), aubergines (Solanum melongena).

“Stone fruit” refers to the plants whose fruits take the form of stone fruit, and to the respective fruits themselves. In these, the inner portion of the fruit wall is ligneous, whereas they are generally fleshy and usually juicy on the outside. These include both the species having edible fruit flesh, especially mangoes (Mangifera genus, for example M. indica), mirabelle plums (Prunus domestica subsp. syriaca), nectarines/peaches (Prunus persica), plums (Prunus domestica), apricots (Prunus armeniaca), olives (Olea europaea), sour cherries (Prunus cerasus) and sweet cherries (Prunus avium), and also the species having edible seeds or edible seed flesh, especially almonds (Prunus dulcis) and pistachios (Pistacia vera). Further stone fruits include the aggregate stone fruits and the plants that form them, especially blackberries (Rubus fruticoruss), raspberries (Rubus idaeus), figs (Ficus carica) and the jackfruit tree (Artocarpus heterophyllus).

Cucurbitaceae are a family of angiosperms. The cucurbitaceae especially include cucumbers (Cucumis sativus), water melons (Citrullus lanatus), melons (Cucumis melo), pumpkins (Cucurbita genus), courgettes (Cucurbita pepo, especially Cucurbita pepo subsp. pepo).

“Cotton” (Gossypium) are plants assigned to the biological genus of Gossypium. These include especially Gossypium barbadense, Gossypium hirsutum and Gossypium herbaceum.

“Nut fruits” refers to the plants whose fruits take the form of nut fruits, and to the respective nut fruits themselves. Nut fruits are non-dehiscent fruits in which all three layers of the fruit wall become ligneous. These especially include walnuts (Juglans genus, for example Junglans regia), sweet chestnuts (Castanea sativa), hazelnuts/common hazel (Corylus avellana) and macadamia nuts (Macadamia genus, for example M. tetraphylla and M. integrifolia). In addition, the nut fruits include the aggregate nut fruits and the plants that form them, especially strawberries (Fragaria genus).

“Crop” in the context of the present application relates to a crop of plants. A crop of plants consists of at least one plant (or plant parts such as fruits) of one of the genera or species specified in this application. A crop in the context of the present application may thus relate to at least one wild plant (or plant parts such as fruits) of one of the genera or species specified in this application; or may preferably relate to at least one crop plant (or plant parts such as fruits), i.e. a plant which has been grown deliberately as a useful or ornamental plant by human intervention and/or processed for breeding purposes. A crop in the context of the present application may be part of a monoculture or part of a mixed culture. A monoculture is a term for the regional concentration of agricultural market production on one product (e.g. green beans, apples, pears, almonds, maize, cotton, soya beans, tomatoes, bell peppers or grapes). Usually, the person skilled in the art understands a monoculture to mean a crop rotation of useful plants consisting only of one useful plant species. A mixed culture may consist of wild plants of various species/genera, but in accordance with the invention encompasses one crop in the context of the present application, or a mixed culture may consist of crop plants of various species/genera, but in accordance with the invention encompasses one crop in the context of the present application.

All the plants and crops mentioned may also be transgenic plants or crops which have originated from the respective plants or crops through genetic modification, for example Bt cotton.

The terms “compound of the formula (I)” and “compound (I)” are interchangeable.

### DETAILED DESCRIPTION

The compound of the formula (I) is of particularly good suitability for use in controlling spider mites from the order of Acari in crops selected from green beans (Phaseolus vulgaris), soya beans (Glycine max), cotton (Gossypium), maize (Zea mays), tea (Camellia sinensis), coffee (Coffea, especially Coffea arabica and Coffea canephora), grapes (Vitis vinifera, preferably Vitis vinifera L. subsp. vinifera), pome fruit, Solanaceae, stone fruit, especially almonds (Prunus dulcis), Cucurbitaceae, and nut fruits, under the condition that the use of the compound of the formula (I) for controlling Tetranychus urticae in green beans (Phaseolus vulgaris) is excluded.

A preferred embodiment is directed to the use of the compound of the formula (I) in the control of spider mites/pests from the Tetranychus genus in the abovementioned crops. Particular preference is given to the use of the compound of the formula (I) in the control of Tetranychus urticae and Tetranychus pacificus.

A further preferred embodiment is directed to the use of the compound of the formula (I) in the control of spider mites/pests from the Oligonychus genus in the abovementioned crops. Particular preference is given to the use of the compound of the formula (I) in the control of Oligonychus pratensis and Oligonychus coffeae.

A further preferred embodiment is directed to the use of the compound of the formula (I) in the control of Polyphagotarsonemus latus.

A further preferred embodiment is directed to the use of the compound of the formula (I) in the control of Panonychus ulmi.

Thus, a preferred embodiment is directed to the use of the compound of the formula (I) in the control of spider mites/pests selected from Tetranychus urticae, Tetranychus pacificus, Oligonychus pratensis, Oligonychus coffeae, Polyphagotarsonemus latus and Panonychus ulmi.

A further preferred embodiment is directed to the use of the compound of the formula (I) in the control of spider mites/pests selected from Tetranychus pacificus, Oligonychus pratensis, Oligonychus coffeae, Polyphagotarsonemus latus and Panonychus ulmi.

A further preferred embodiment is directed to the use of the compound of the formula (I) in the control of spider mites/pests from the Tetranychidae family, selected from Tetranychus urticae.

A further preferred embodiment is directed to the use of the compound of the formula (I) in the control of spider mites/pests selected from Oligonychus pratensis, Oligonychus coffeae, Polyphagotarsonemus latus and Panonychus ulmi.

A further preferred embodiment is directed to the use of the compound of the formula (I) in the control of spider mites/pests from the Acari order, selected from Tetranychus urticae, Tetranychus pacificus, Oligonychus pratensis, Oligonychus coffeae, Polyphagotarsonemus latus and Panonychus ulmi, in crops selected from soya beans (Glycine max), cotton (Gossypium), maize (Zea mays), tea (Camellia sinensis), coffee (Coffea, preferably Coffea arabica and Coffea canephora), grapes (Vitis vinifera, preferably Vitis vinifera L. subsp. vinifera), pome fruit, Solanaceae, stone fruit, especially almonds (Prunus dulcis), Cucurbitaceae, and nut fruits.

A further preferred embodiment is directed to the use of the compound of the formula (I) in the control of spider mites/pests from the Acari order, selected from Tetranychus pacificus, Oligonychus pratensis, Oligonychus coffeae, Polyphagotarsonemus latus and Panonychus ulmi, in crops selected from green beans (Phaseolus vulgaris), soya beans (Glycine max), cotton (Gossypium), maize (Zea mays), tea (Camellia sinensis), coffee (Coffea, preferably Coffea arabica and Coffea canephora), grapes (Vitis vinifera, preferably Vitis vinifera L. subsp. vinifera), pome fruit, Solanaceae, stone fruit, especially almonds (Prunus dulcis), Cucurbitaceae, and nut fruits.

A further preferred embodiment is directed to the use of the compound of the formula (I) in the control of spider mites/pests from the Acari order, selected from Tetranychus pacificus, Oligonychus pratensis, Oligonychus coffeae, Polyphagotarsonemus latus and Panonychus ulmi, in crops selected from green beans (Phaseolus vulgaris), soya beans (Glycine max), cotton (Gossypium), maize (Zea mays), tea (Camellia sinensis), coffee (Coffea, preferably Coffea arabica and Coffea canephora), grapes (Vitis vinifera, preferably Vitis vinifera L. subsp. vinifera), pome fruit, Solanaceae, almonds (Prunus dulcis), Cucurbitaceae.

The following combinations of pests/spider mites and crop are particularly preferred embodiments of the inventive use of the compound of the formula (I) for control of spider mites in particular crops:

Tetranychus in green beans (Phaseolus vulgaris), soya beans (Glycine max), cotton (Gossypium), maize (Zea mays), tea (Camellia sinensis), coffee (Coffea, preferably Coffea arabica and Coffea canephora), grapes (Vitis vinifera, preferably Vitis vinifera L. subsp. vinifera), pome fruit, preferably apples and pears, Solanaceae, stone fruit, preferably almonds (Prunus dulcis), Cucurbitaceae, and nut fruits, under the condition that the use of the compound of the formula (I) for controlling Tetranychus urticae in green beans (Phaseolus vulgaris) is excluded.

Tetranychus in green beans (Phaseolus vulgaris), excluding Tetranychus urticae,

Tetranychus in soya beans (Glycine max),

Tetranychus in cotton (Gossypium),

Tetranychus in maize (Zea mays),

Tetranychus in tea (Camellia sinensis),

Tetranychus in coffee (Coffea, preferably Coffea arabica and Coffea canephora),

Tetranychus in grapes (Vitis vinifera, preferably Vitis vinifera L. subsp. vinifera),

Tetranychus in pome fruit, preferably apples and pears,

Tetranychus in Solanaceae,

Tetranychus in stone fruit, preferably almonds (Prunus dulcis),

Tetranychus in Cucurbitaceae,

Tetranychus in nut fruits,

Tetranvchus urticae

Tetranychus urticae in green beans (Phaseolus vulgaris), soya beans (Glycine max), cotton (Gossypium), maize (Zea mays), tea (Camellia sinensis), coffee (Coffea, preferably Coffea arabica and Coffea canephora), grapes (Vitis vinifera, preferably Vitis vinifera L. subsp. vinifera), pome fruit, preferably apples and pears, Solanaceae, stone fruit, preferably almonds (Prunus dulcis), Cucurbitaceae, and nut fruits,

Tetranychus urticae in soya beans (Glycine max),

Tetranychus urticae in cotton (Gossypium),

Tetranychus urticae in maize (Zea mays),

Tetranychus urticae in tea (Camellia sinensis),

Tetranychus urticae in coffee (Coffea, preferably Coffea arabica and Coffea canephora),

Tetranychus urticae in grapes (Vitis vinifera, preferably Vitis vinifera L. subsp. vinifera),

Tetranychus urticae in pome fruit, preferably apples and pears,

Tetranychus urticae in Solanaceae,

Tetranychus urticae in stone fruit, preferably almonds (Prunus dulcis),

Tetranychus urticae in Cucurbitaceae,

Tetranychus urticae in nut fruits,

Especially preferred is the inventive control of Tetranychus urticae in soya beans, maize or cotton;

Tetranychus pacificus

Tetranychus pacificus in green beans (Phaseolus vulgaris), soya beans (Glycine max), cotton (Gossypium), maize (Zea mays), tea (Camellia sinensis), coffee (Coffea, preferably Coffea arabica and Coffea canephora), grapes (Vitis vinifera, preferably Vitis vinifera L. subsp. vinifera), pome fruit, preferably apples and pears, Solanaceae, stone fruit, preferably almonds (Prunus dulcis), Cucurbitaceae, and nut fruits,

Tetranychus pacificus in green beans (Phaseolus vulgaris),

Tetranychus pacificus in soya beans (Glycine max),

Tetranychus pacificus in cotton (Gossypium),

Tetranychus pacificus in maize (Zea mays),

Tetranychus pacificus in tea (Camellia sinensis),

Tetranychus pacificus in coffee (Coffea, preferably Coffea arabica and Coffea canephora),

Tetranychus pacificus in grapes (Vitis vinifera, preferably Vitis vinifera L. subsp. vinifera),

Tetranychus pacificus in pome fruit, preferably apples and pears,

Tetranychus pacificus in Solanaceae,

Tetranychus pacificus in stone fruit, preferably almonds (Prunus dulcis),

Tetranychus pacificus in Cucurbitaceae,

Tetranychus pacificus in nut fruits,

Especially preferred is the inventive control of Tetranychus pacificus in stone fruit or grapes (Vitis vinifera, preferably Vitis vinifera L. subsp. vinifera); more preferably in almonds or grapes (Vitis vinifera, preferably Vitis vinifera L. subsp. vinifera).

### Oligonychus

Oligonychus in green beans (Phaseolus vulgaris), soya beans (Glycine max), cotton (Gossypium), maize (Zea mays), tea (Camellia sinensis), coffee (Coffea, preferably Coffea arabica and Coffea canephora), grapes (Vitis vinifera, preferably Vitis vinifera L. subsp. vinifera), pome fruit, preferably apples and pears, Solanaceae, stone fruit, preferably almonds (Prunus dulcis), Cucurbitaceae, and nut fruits,

Oligonychus in green beans (Phaseolus vulgaris),

Oligonychus in soya beans (Glycine max),

Oligonychus in cotton (Gossypium),

Oligonychus in maize (Zea mays),

Oligonychus in tea (Camellia sinensis),

Oligonychus in coffee (Coffea, preferably Coffea arabica and Coffea canephora),

Oligonychus in grapes (Vitis vinifera, preferably Vitis vinifera L. subsp. vinifera),

Oligonychus in pome fruit, preferably apples and pears,

Oligonychus in Solanaceae,

Oligonychus in stone fruit, preferably almonds (Prunus dulcis),

Oligonychus in Cucurbitaceae,

Oligonychus in nut fruits.

Oligonychus pratensis

Oligonychus pratensis in green beans (Phaseolus vulgaris), soya beans (Glycine max), cotton (Gossypium), maize (Zea mays), tea (Camellia sinensis), coffee (Coffea, preferably Coffea arabica and Coffea canephora), grapes (Vitis vinifera, preferably Vitis vinifera L. subsp. vinifera), pome fruit, preferably apples and pears, Solanaceae, stone fruit, preferably almonds (Prunus dulcis), Cucurbitaceae, and nut fruits,

Oligonychus pratensis in green beans (Phaseolus vulgaris),

Oligonychus pratensis in soya beans (Glycine max),

Oligonychus pratensis in cotton (Gossypium),

Oligonychus pratensis in maize (Zea mays),

Oligonychus pratensis in tea (Camellia sinensis),

Oligonychus pratensis in coffee (Coffea, preferably Coffea arabica and Coffea canephora),

Oligonychus pratensis in grapes (Vitis vinifera, preferably Vitis vinifera L. subsp. vinifera),

Oligonychus pratensis in pome fruit, preferably apples and pears,

Oligonychus pratensis in Solanaceae,

Oligonychus pratensis in stone fruit, preferably almonds (Prunus dulcis),

Oligonychus pratensis in Cucurbitaceae,

Oligonychus pratensis in nut fruits,

Especially preferred is the inventive control of Oligonychus pratensis in maize

Oligonvchus coffeae

Oligonychus coffeae in green beans (Phaseolus vulgaris), soya beans (Glycine max), cotton (Gossypium), maize (Zea mays), tea (Camellia sinensis), coffee (Coffea, preferably Coffea arabica and Coffea canephora), grapes (Vitis vinifera, preferably Vitis vinifera L. subsp. vinifera), pome fruit, preferably apples and pears, Solanaceae, stone fruit, preferably almonds (Prunus dulcis), Cucurbitaceae, and nut fruits,

Oligonychus coffeae in green beans (Phaseolus vulgaris),

Oligonychus coffeae in soya beans (Glycine max),

Oligonychus coffeae in cotton (Gossypium),

Oligonychus coffeae in maize (Zea mays),

Oligonychus coffeae in tea (Camellia sinensis),

Oligonychus coffeae in coffee (Coffea, preferably Coffea arabica and Coffea canephora),

Oligonychus coffeae in grapes (Vitis vinifera, preferably Vitis vinifera L. subsp. vinifera),

Oligonychus coffeae in pome fruit, preferably apples and pears,

Oligonychus coffeae in Solanaceae,

Oligonychus coffeae in stone fruit, preferably almonds (Prunus dulcis),

Oligonychus coffeae in Cucurbitaceae,

Oligonychus coffeae in nut fruits,

Especially preferred is the inventive control of Oligonychus coffeae in tea or in coffee (Coffea, preferably Coffea arabica and Coffea canephora).

Polyphagotarsonemus latus

Polyphagotarsonemus latus in green beans (Phaseolus vulgaris), soya beans (Glycine max), cotton (Gossypium), maize (Zea mays), tea (Camellia sinensis), coffee (Coffea, preferably Coffea arabica and Coffea canephora),

grapes (Vitis vinifera, preferably Vitis vinifera L. subsp. vinifera), pome fruit, preferably apples and pears, Solanaceae, stone fruit, preferably almonds (Prunus dulcis), Cucurbitaceae, and nut fruits,

Polyphagotarsonemus latus in green beans (Phaseolus vulgaris),

Polyphagotarsonemus latus in soya beans (Glycine max),

Polyphagotarsonemus latus in cotton (Gossypium),

Polyphagotarsonemus latus in maize (Zea mays),

Polyphagotarsonemus latus in tea (Camellia sinensis),

Polyphagotarsonemus latus in coffee (Coffea, preferably Coffea arabica and Coffea canephora),

Polyphagotarsonemus latus in grapes (Vitis vinifera, preferably Vitis vinifera L. subsp. vinifera),

Polyphagotarsonemus latus in pome fruit, preferably apples and pears,

Polyphagotarsonemus latus in Solanaceae,

Polyphagotarsonemus latus in stone fruit, preferably almonds (Prunus dulcis),

Polyphagotarsonemus latus in Cucurbitaceae,

Polyphagotarsonemus latus in nut fruits,

Polyphagotarsonemus latus in grapes (Vitis vinifera, preferably Vitis vinifera L. subsp. vinifera),

Polyphagotarsonemus latus in nut fruits,

Especially preferred is the inventive control of Polyphagotarsonemus latus in green beans (Phaseolus vulgaris), cotton, grapes (Vitis vinifera, preferably Vitis vinifera L. subsp. vinifera), Solanaceae (preferably tomatoes (S. lycospersicum), bell pepper species (Capsicum genus), aubergines (Solanum melongena)), pome fruit (preferably apples and pears) or Cucurbitaceae, more preferably in a crop selected from the group consisting of green beans (Phaseolus vulgaris), cotton and Solanaceae (preferably tomatoes (S. lycospersicum), bell pepper species (Capsicum genus), aubergines (Solanum melongena)).

Panonvchus ulmi

Panonychus ulmi in green beans (Phaseolus vulgaris), soya beans (Glycine max), cotton (Gossypium), maize (Zea mays), tea (Camellia sinensis), coffee (Coffea, preferably Coffea arabica and Coffea canephora), grapes (Vitis vinifera, preferably Vitis vinifera L. subsp. vinifera), pome fruit, preferably apples and pears, Solanaceae, stone fruit, preferably almonds (Prunus dulcis), Cucurbitaceae, and nut fruits,

Panonychus ulmi in green beans (Phaseolus vulgaris),

Panonychus ulmi in soya beans (Glycine max),

Panonychus ulmi in cotton (Gossypium),

Panonychus ulmi in maize (Zea mays),

Panonychus ulmi in tea (Camellia sinensis),

Panonychus ulmi in coffee (Coffea, preferably Coffea arabica and Coffea canephora),

Panonychus ulmi in grapes (Vitis vinifera, preferably Vitis vinifera L. subsp. vinifera),

Panonychus ulmi in pome fruit, preferably apples and pears,

Panonychus ulmi in Solanaceae,

Panonychus ulmi in stone fruit, preferably almonds (Prunus dulcis),

Panonychus ulmi in Cucurbitaceae,

Panonychus ulmi in nut fruits,

Panonychus ulmi in grapes (Vitis vinifera, preferably Vitis vinifera L. subsp. vinifera),

Panonychus ulmi in nut fruits,

Especially preferred is the inventive control of Panonychus ulmi in grapes (Vitis vinifera, preferably Vitis vimfera subsp. vinifera) or pome fruit, most preferably in grapes (Vitis vinifera, preferably Vitis vinifera subsp. vinifera), apples or pears.

### Formulations

The present invention further relates to the inventive use of the compound of the formula (I) in the form of a formulation. The compound of the formula (I) can be used in various use forms for the inventive use, for example drench, drip and spray liquors, comprising the compound of the formula (I). In some cases, the use forms comprise further pesticides and/or adjuvants which improve action, such as penetrants, e.g. vegetable oils, for example rapeseed oil, sunflower oil, mineral oils, for example paraffin oils, alkyl esters of vegetable fatty acids, for example rapeseed oil methyl ester or soya oil methyl ester, or alkanol alkoxylates and/or spreaders, for example alkylsiloxanes and/or salts, for example organic or inorganic ammonium or phosphonium salts, for example ammonium sulphate or diammonium hydrogenphosphate and/or retention promoters, for example dioctyl sulphosuccinate or hydroxypropylguar polymers and/or humectants, for example glycerol and/or fertilizers, for example ammonium-, potassium- or phosphorus-containing fertilizers.

Customary formulations are, for example, water-soluble liquids (SL), emulsion concentrates (EC), emulsions in water (EW), suspension concentrates (SC, SE, FS, OD), water-dispersible granules (WG), granules (GR) and capsule concentrates (CS); these and further possible formulation types are described, for example, by Crop Life International and in Pesticide Specifications, Manual on development and use of FAO and WHO specifications for pesticides, FAO Plant Production and Protection Papers-13 173, prepared by the FAO/WHO Joint Meeting on Pesticide Specifications, 2004, ISBN: 9251048576. The formulations, in addition to one or more compounds of the formula (I), optionally comprise further agrochemically active ingredients.

These are preferably formulations or use forms which comprise auxiliaries, for example extenders, solvents, spontaneity promoters, carriers, emulsifiers, dispersants, frost protectants, biocides, thickeners and/or further auxiliaries, for example adjuvants. An adjuvant in this context is a component which enhances the biological effect of the formulation, without the component itself having any biological effect. Examples of adjuvants are agents which promote retention, spreading, attachment to the leaf surface or penetration.

These formulations are produced in a known manner, for example by mixing the compound of the formula (I) with auxiliaries, for example extenders, solvents and/or solid carriers and/or other auxiliaries, for example surfactants. The formulations are produced either in suitable facilities or else before or during application.

Auxiliaries used may be substances suitable for imparting special properties, such as certain physical, technical and/or biological properties, to the formulation of the compound of the formula (I), or to the use forms prepared from these formulations (for example ready-to-use pesticides such as spray liquors or seed dressing products).

Suitable extenders are, for example, water, polar and nonpolar organic chemical liquids, for example from the classes of the aromatic and non-aromatic hydrocarbons (such as paraffins, alkylbenzenes, alkylnaphthalenes, chlorobenzenes), the alcohols and polyols (which, if appropriate, may also be substituted, etherified and/or esterified), the ketones (such as acetone, cyclohexanone), esters (including fats and oils) and (poly)ethers, the unsubstituted and substituted amines, amides, lactams (such as N-alkylpyrrolidones) and lactones, the sulphones and sulphoxides (such as dimethyl sulphoxide).

If the extender utilized is water, it is also possible to use, for example, organic solvents as auxiliary solvents. Useful liquid solvents are essentially: aromatics such as xylene, toluene or alkylnaphthalenes, chlorinated aromatics or chlorinated aliphatic hydrocarbons such as chlorobenzenes, chloroethylenes or methylene chloride, aliphatic hydrocarbons such as cyclohexane or paraffins, for example mineral oil fractions, mineral and vegetable oils, alcohols such as butanol or glycol and their ethers and esters, ketones such as acetone, methyl ethyl ketone, methyl isobutyl ketone or cyclohexanone, strongly polar solvents such as dimethylformamide and dimethyl sulphoxide, and also water.

In principle, it is possible to use all suitable solvents. Examples of suitable solvents are aromatic hydrocarbons, such as xylene, toluene or alkylnaphthalenes, chlorinated aromatic or aliphatic hydrocarbons, such as chlorobenzene, chloroethylene or methylene chloride, aliphatic hydrocarbons, such as cyclohexane, paraffins, mineral oil fractions, mineral and vegetable oils, alcohols, such as methanol, ethanol, isopropanol, butanol or glycol and their ethers and esters, ketones such as acetone, methyl ethyl ketone, methyl isobutyl ketone or cyclohexanone, strongly polar solvents, such as dimethyl sulphoxide, and also water.

In principle, it is possible to use all suitable carriers. Useful carriers especially include, for example, ammonium salts and natural, finely ground rocks, such as kaolins, aluminas, talc, chalk, quartz, attapulgite, montmorillonite or diatomaceous earth, and synthetic, finely ground rocks, such as highly disperse silica, aluminium oxide and natural or synthetic silicates, resins, waxes and/or solid fertilizers. Mixtures of such carriers can likewise be used. Useful carriers for granules include: for example crushed and fractionated natural rocks such as calcite, marble, pumice, sepiolite, dolomite, and synthetic granules of inorganic and organic flours, and also granules of organic material such as sawdust, paper, coconut shells, corn cobs and tobacco stalks.

Liquefied gaseous extenders or solvents can also be used. Especially suitable are those extenders or carriers which are gaseous at standard temperature and under standard pressure, for example aerosol propellants such as halohydrocarbons, or else butane, propane, nitrogen and carbon dioxide.

Examples of emulsifiers and/or foam formers, dispersants or wetting agents with ionic or nonionic properties, or mixtures of these surfactants, are salts of polyacrylic acid, salts of lignosulphonic acid, salts of phenolsulphonic acid or naphthalenesulphonic acid, polycondensates of ethylene oxide with fatty alcohols or with fatty acids or with fatty amines, with substituted phenols (preferably alkylphenols or arylphenols), salts of sulphosuccinic esters, taurine derivatives (preferably alkyl taurates), phosphoric esters of polyethoxylated alcohols or phenols, fatty acid esters of polyols, and derivatives of the compounds containing sulphates, sulphonates and phosphates, for example alkylaryl polyglycol ethers, alkyl sulphonates, alkyl sulphates, arylsulphonates, protein hydrolysates, lignosulphite waste liquors and methylcellulose. The presence of a surfactant is advantageous when one of the compounds of the formula (I) and/or one of the inert carriers is insoluble in water and when the application takes place in water.

Further auxiliaries which may be present in the formulations and the use forms derived therefrom include dyes such as inorganic pigments, for example iron oxide, titanium oxide and Prussian Blue, and organic dyes such as alizarin dyes, azo dyes and metal phthalocyanine dyes, and nutrients and trace nutrients such as salts of iron, manganese, boron, copper, cobalt, molybdenum and zinc.

Additional components may be stabilizers, such as cold stabilizers, preservatives, antioxidants, light stabilizers, or other agents which improve chemical and/or physical stability. Foam generators or antifoams may also be present.

In addition, the formulations and the use forms derived therefrom may also comprise, as additional auxiliaries, stickers such as carboxymethyl cellulose and natural and synthetic polymers in the form of powders, granules or latices, such as gum arabic, polyvinyl alcohol and polyvinyl acetate, or else natural phospholipids such as cephalins and lecithins and synthetic phospholipids. Further possible auxiliaries are mineral and vegetable oils.

Optionally, further auxiliaries may be present in the formulations and the use forms derived therefrom. Examples of such additives are fragrances, protective colloids, binders, adhesives, thickeners, thixotropic agents, penetrants, retention promoters, stabilizers, sequestrants, complexing agents, humectants, spreaders. In general, the compounds of the formula (I) can be combined with any solid or liquid additive commonly used for formulation purposes.

Useful retention promoters include all those substances which reduce the dynamic surface tension, for example dioctyl sulphosuccinate, or increase the viscoelasticity, for example hydroxypropylguar polymers.

Useful penetrants in the present context are all those substances which are typically used to improve the penetration of active agrochemical ingredients into plants. Penetrants are defined in this context by their ability to penetrate from the (generally aqueous) application liquor and/or from the spray coating into the cuticle of the plant and hence increase the mobility of active ingredients in the cuticle. The method described in the literature (Baur et al., 1997, Pesticide Science 51, 131-152) can be used for determining this property. Examples include alcohol alkoxylates, for example coconut fatty ethoxylate (10) or isotridecyl ethoxylate (12), fatty acid esters, for example rapeseed oil methyl ester or soya oil methyl ester, fatty amine alkoxylates, for example tallowamine ethoxylate (15), or ammonium and/or phosphonium salts, for example ammonium sulphate or diammonium hydrogenphosphate.

The formulations preferably contain between 0.00000001% and 98% by weight of the compound of the formula (I), more preferably between 0.01% and 95% by weight of the compound of the formula (I), most preferably between 0.5% and 90% by weight of the compound of the formula (I), based on the weight of the formulation.

The content of the compound of the formula (I) in the use forms prepared from the formulations (especially pesticides) may vary within wide ranges. The concentration of the compound of the formula (I) in the use forms may typically be between 0.00000001% and 95% by weight of the compound of the formula (I), preferably between 0.00001% and 1% by weight, based on the weight of the use form. Application is accomplished in a customary manner appropriate for the use forms.

### Mixtures

The compound of the formula (I) can also be used, on its own or in formulations thereof, in a mixture with one or more suitable fungicides, bactericides, acaricides, molluscicides, nematicides, insecticides, microbiologicals, beneficial organisms, herbicides, fertilizers, bird repellents, phytotonics, sterilants, synergists, safeners, semiochemicals and/or plant growth regulators, in order thus, for example, to broaden the spectrum of action, to prolong the duration of action, to increase the rate of action, to prevent repulsion or prevent evolution of resistance. In addition, active ingredient combinations of this kind can improve plant growth and/or tolerance to abiotic factors, for example high or low temperatures, to drought or to elevated water content or soil salinity. It is also possible to improve flowering and fruiting performance, optimize germination capacity and root development, facilitate harvesting and improve yields, influence maturation, improve the quality and/or the nutritional value of the harvested products, prolong storage life and/or improve the processability of the harvested products.

In addition, the compound of the formula (I), on its own or in (commercial) formulations thereof and in the use forms prepared from these formulations, may be present in a mixture with further active ingredients or semiochemicals, such as attractants and/or bird repellents and/or plant activators and/or growth regulators and/or fertilizers and/or synergists. It is likewise possible to use the compound of the formula (I) and compositions thereof in mixtures with agents to improve plant properties, for example growth, yield and quality of the harvested material.

### Plants and Plant Parts

In the inventive use, it is possible to treat all plants and parts of plants with a compound of the formula (I). Plants are understood here to mean all plants and plant populations, such as desirable and undesirable wild plants or crop plants (including naturally occurring crop plants), for example maize, soya, tomatoes and other vegetables, cotton, and fruit plants (yielding apples, pears, and grapes or stone fruit, for example almonds). Crop plants may be plants which can be obtained by conventional breeding and optimization methods or by biotechnological and genetic engineering methods or combinations of these methods, including the transgenic plants and including the plant cultivars which are protectable or non-protectable by plant breeders' rights. Parts of plants shall be understood to mean all parts and organs of the plants above and below ground, such as shoot, leaf, flower and root, examples given being leaves, needles, stalks, stems, flowers, fruit bodies, fruits and seeds, and also roots, tubers and rhizomes. Parts of plants also include harvested material and vegetative and generative propagation material, for example cuttings, tubers, rhizomes, slips and seeds.

For the inventive use with the compound of the formula (I), the treatment of the plants and parts of plants with the compound of the formula (I) is effected directly or by allowing it to act on the surroundings, habitat or storage space thereof by the customary treatment methods, for example by dipping, spraying, evaporating, fogging, scattering, painting on, injecting, and, in the case of propagation material, especially in the case of seeds, also by applying one or more coats.

As already mentioned above, it is possible in accordance with the invention to treat all plants and parts thereof. In a preferred embodiment, wild plant species and plant cultivars, or those obtained by conventional biological breeding methods, such as crossing or protoplast fusion, and parts thereof, are treated. In a further preferred embodiment, transgenic plants and plant cultivars obtained by genetic engineering methods, if appropriate in combination with conventional methods (genetically modified organisms), and parts thereof are treated. The term “parts” or “parts of plants” or “plant parts” has been explained above. Particular preference is given in accordance with the invention to treating plants of the respective commercially customary plant cultivars or those that are in use. Plant cultivars are understood to mean plants having new properties (“traits”) and which have been grown by conventional breeding, by mutagenesis or by recombinant DNA techniques. They may be cultivars, varieties, biotypes or genotypes. The transgenic plants especially include Bt plants, which generate Bt toxins in the plants by virtue of the genetic material from Bacillus thuringiensis (for example by virtue of the CryIA(a), CryIA(b), CryIA(c), CryIIA, CryIIIA, CryIIIB2, Cry9c, Cry2Ab, Cry3Bb and CryIF genes and combinations thereof). Preference is given to plants containing the CryIA(a), CryIA(b), CryIA(c) genes. The genes which impart the desired properties (“traits”) in question may also be present in combinations with one another in the transgenic plants.

The good efficacy of the compound of the formula (I) against pests from the family of the spider mites (Tetranychidae) in appropriate crops is apparent from the following examples:

### EXAMPLE 1

Tetranvchus urticae Test; Outdoor Spray Treatment (TETRUR)

To produce an appropriate active ingredient formulation, 1 part by weight of active ingredient is mixed with the stated amounts of water to the desired concentration. Added to the spray liquor is 0.1% a.i. Crovol.

Three aubergine plants (Solanum melongena) infested by all stages of the red spider mite (Tetranychus urticae) are sprayed with an active ingredient formulation of each desired concentration. One spray application is conducted in each case.

After the desired time, the remaining mites per leaf are counted and calculated as the efficacy by the Abbott formula:

$$Efficacy \% = ( 1 - \frac{number in B after treatment}{number in K after treatment} ) \times 100$$

$$B = treated plots$$

$$K = untreated control plots$$

In this test, the compound tested shows good efficacy compared to the spirodiclofen standard used:

| | | Animal | | Concentration | Efficacy | |
|---|---|---|---|---|---|---|
| Substance | Structure | species | Plant | (g of ai/ha) | (% Abbott) | Dat * |
| Spirodiclofen | 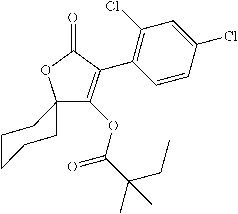 [SMILES: CCC(C)(C)C(=O)OC1=C(c2ccc(Cl)cc2Cl)C(=O)OC12CCCCC2] | TETRUR | S. melongena | 48 | 63 89 97.5 95 80 | 5 7 14 21 29 |
| Compound (I) (+Crovol 0.1%) | 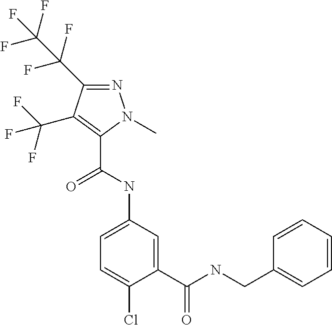 [SMILES: Cn1nc(C(F)(F)C(F)(F)F)c(C(F)(F)F)c1C(=O)Nc1ccc(Cl)c(C(=O)NCc2ccccc2)c1] | TETRUR | S. melongena | 100 | 99 100 100 99 99 | 5 7 14 21 29 |

* Dat = days after treatment

### EXAMPLE 2

Tetranvchus urticae Test; Outdoor Spray Treatment (TETRUR)

To produce an appropriate active ingredient formulation, 1 part by weight of active ingredient is mixed with the stated amounts of water to the desired concentration. Added to the spray liquor is 0.1% a.i. Crovol.

Three cotton plants (Gossypium hirsutum) infested by all stages of the red spider mite (Tetranychus urticae) are sprayed with an active ingredient formulation of each desired concentration. One spray application is conducted in each case.

After the desired time, the remaining mites per leaf are counted and calculated as the efficacy by the Abbott formula:

$$Efficacy \% = ( 1 - \frac{number in B after treatment}{number in K after treatment} ) \times 100$$

B=treated plots
K=untreated control plots

In this test, the compound tested shows good efficacy compared to the cyenopyrafen standard used:

| | | Animal | | Concentration | Efficacy | |
|---|---|---|---|---|---|---|
| Substance | Structure | species | Plant | (g of ai/ha) | (% Abbott) | Dat * |
| Cyenopyrafen | 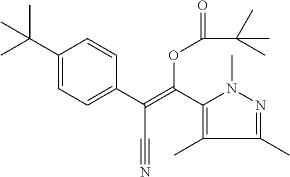 [SMILES: Cc1nn(C)c(/C(OC(=O)C(C)(C)C)=C(\C#N)c2ccc(C(C)(C)C)cc2)c1C] | TETRUR | G. hirsutum | 150 | 98 99 | 4 6 |
| Compound (I) (+Crovol 0.1%) | 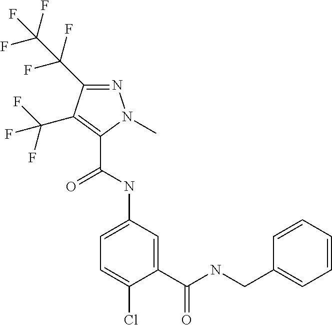 [SMILES: Cn1nc(C(F)(F)C(F)(F)F)c(C(F)(F)F)c1C(=O)Nc1ccc(Cl)c(C(=O)NCc2ccccc2)c1] | TETRUR | G. hirsutum | 100 | 100 100 | 4 6 |

* Dat =days after treatment

### EXAMPLE 3

Polyphagotarsonemus latus Test; Outdoor Spray Treatment (HEMTLA)

To produce an appropriate active ingredient formulation, 1 part by weight of active ingredient is mixed with the stated amounts of water to the desired concentration. Added to the spray liquor is 0.1% a.i. Crovol.

Three bean plants (Phaseolus vulgaris) infested by all stages of the broad mite (Polyphagotarsonemus latus) are sprayed with an active ingredient formulation of each desired concentration. Each plant is treated three times at 7-day intervals with a spray application with a water application rate of 200 l/Ha. The juncture of treatment of the plants with the first spray application X corresponds to the start point of the evaluation (Dat=0) (Dat=days after treatment). The further treatments were effected after 7 days (spray application Y), the third treatment after 14 days (spray application Z).

After the desired time, the remaining mites per leaf are counted and calculated as the efficacy by the Abbott formula:

$$Efficacy \% = ( 1 - \frac{number in B after treatment}{number in K after treatment} ) \times 100$$

$$B = treated plots$$

$$K = untreated control plots$$

In this test, the compound tested shows good efficacy compared to the cyenopyrafen standard used:

| | | Animal | | Concentration | Efficacy | |
|---|---|---|---|---|---|---|
| Substance | Structure | species | Plant | (g of ai/ha) | (% Abbott) | Dat * |
| Cyenopyrafen | 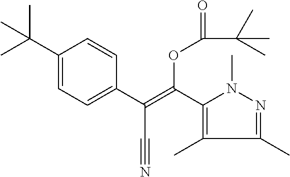 [SMILES: Cc1nn(C)c(/C(OC(=O)C(C)(C)C)=C(\C#N)c2ccc(C(C)(C)C)cc2)c1C] | HEMTLA | Ph. vulgaris | 150 | 83.7 63.8 48.5 58.1 0 | 3 7 (Y) 14 (Z) 21 28 |
| Compound (I) (+Crovol 0.1%) | 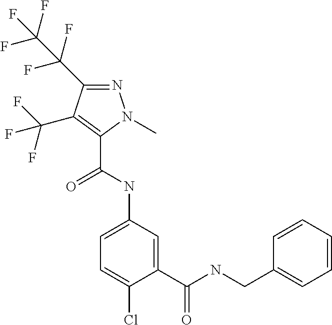 [SMILES: Cn1nc(C(F)(F)C(F)(F)F)c(C(F)(F)F)c1C(=O)Nc1ccc(Cl)c(C(=O)NCc2ccccc2)c1] | HEMTLA | Ph. vulgaris | 100 75 | 81.4 91.5 90 91.9 52.7 72 80.7 81.2 79.2 49.7 | 3 7 (Y) 14 (Z) 21 28 3 7 14 (Y) 21 (Z) 28 |

* Dat = days after treatment with spray application

### EXAMPLE 4

Polyphagotarsonemus latus Test; Outdoor Spray Treatment (HEMTLA)

To produce an appropriate active ingredient formulation, 1 part by weight of active ingredient is mixed with the stated amounts of water to the desired concentration. Added to the spray liquor is 0.1% a.i. Crovol.

Three chilli plants (Capsicum frutescens) infested by all stages of the broad mite (Polyphagotarsonemus latus) are sprayed with an active ingredient formulation of each desired concentration. A spray application with a water application rate of 458 l/Ha is conducted in each case.

After the desired time, the remaining mites per leaf are counted and calculated as the efficacy by the Abbott formula:

$$Efficacy \% = ( 1 - \frac{number in B after treatment}{number in K after treatment} ) \times 100$$

$$B = treated plots$$

$$K = untreated control plots$$

In this test, the compound tested shows good efficacy compared to the cyenopyrafen standard used:

| | | Animal | | Concentration | Efficacy | |
|---|---|---|---|---|---|---|
| Substance | Structure | species | Plant | (g of ai/ha) | (% Abbott) | Dat * |
| Cyenopyrafen | 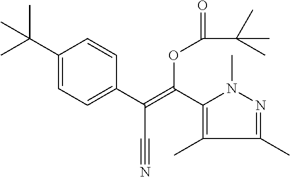 [SMILES: Cc1nn(C)c(/C(OC(=O)C(C)(C)C)=C(\C#N)c2ccc(C(C)(C)C)cc2)c1C] | HEMTLA | C. frutescens | 150 | 28 45 0 | 7 14 21 |
| Compound (I) (+Crovol 0.1%) | 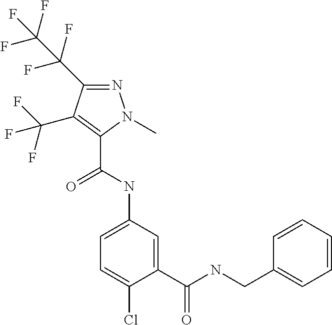 [SMILES: Cn1nc(C(F)(F)C(F)(F)F)c(C(F)(F)F)c1C(=O)Nc1ccc(Cl)c(C(=O)NCc2ccccc2)c1] | HEMTLA | C. frutescens | 100 75 | 75 75 83 63 67 50 | 7 14 21 7 14 21 |

* Dat = days after treatment

### EXAMPLE 5

Polyphagotarsonemus latus Test; Outdoor Spray Treatment (HEMTLA)

To produce an appropriate active ingredient formulation, 1 part by weight of active ingredient is mixed with the stated amounts of water to the desired concentration. Added to the spray liquor is 0.1% a.i. Crovol.

Three aubergine plants (Solanum melongena) infested by all stages of the broad mite (Polyphagotarsonemus latus) are sprayed with an active ingredient formulation of each desired concentration. A spray application with a water application rate of 500 l/Ha is conducted in each case.

After the desired time, the remaining mites per leaf are counted and calculated as the efficacy by the Abbott formula:

$$Efficacy \% = ( 1 - \frac{number in B after treatment}{number in K after treatment} ) \times 100$$

$$B = treated plots$$

$$K = untreated control plots$$

In this test, the compound tested shows good efficacy compared to the fenazaquin standard used:

| | | Animal | | Concentration | Efficacy | |
|---|---|---|---|---|---|---|
| Substance | Structure | species | Plant | (g of ai/ha) | (% Abbott) | Dat * |
| Fenazaquin | 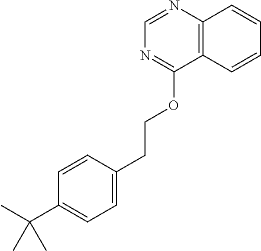 [SMILES: CC(C)(C)c1ccc(CCOc2ncnc3ccccc23)cc1] | HEMTLA | S. melongena | 100 | 97.9 | 16 |
| Compound (I) (+Crovol 0.1%) | 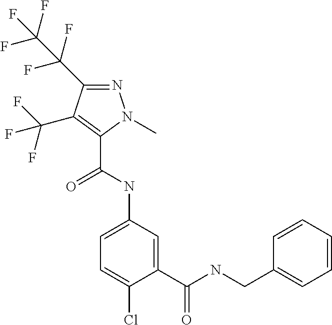 [SMILES: Cn1nc(C(F)(F)C(F)(F)F)c(C(F)(F)F)c1C(=O)Nc1ccc(Cl)c(C(=O)NCc2ccccc2)c1] | HEMTLA | S. melongena | 100 | 100 | 16 |

* Dat = days after treatment

### EXAMPLE 6

Polyphagotarsonemus latus Test; Outdoor Spray Treatment (HEMTLA)

To produce an appropriate active ingredient formulation, 1 part by weight of active ingredient is mixed with the stated amounts of water to the desired concentration. Added to the spray liquor is 0.1% a.i. Crovol.

Three bell pepper plants (Capsicum annuum) infested by all stages of the broad mite (Polyphagotarsonemus latus) are sprayed with an active ingredient formulation of each desired concentration. A spray application with a water application rate of 500 l/Ha is conducted in each case.

After the desired time, the remaining mites per leaf are counted and calculated as the efficacy by the Abbott formula:

$$Efficacy \% = ( 1 - \frac{number in B after treatment}{number in K after treatment} ) \times 100$$

$$B = treated plots$$

$$K = untreated control plots$$

In this test, the compound tested shows good efficacy compared to the fenazaquin standard used:

| | | Animal | | Concentration | Efficacy | |
|---|---|---|---|---|---|---|
| Substance | Structure | species | Plant | (g of ai/ha) | (% Abbott) | Dat * |
| Fenazaquin | 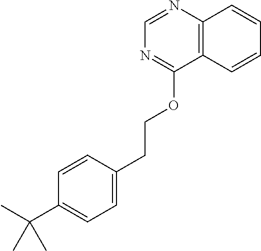 [SMILES: CC(C)(C)c1ccc(CCOc2ncnc3ccccc23)cc1] | HEMTLA | Capsicum annuum | 100 | 92.3 96 77.4 33.5 | 3 7 15 21 |
| Compound (I) (+Crovol 0.1%) | 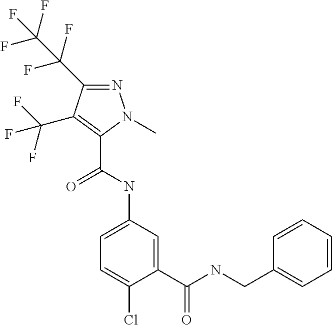 [SMILES: Cn1nc(C(F)(F)C(F)(F)F)c(C(F)(F)F)c1C(=O)Nc1ccc(Cl)c(C(=O)NCc2ccccc2)c1] | HEMTLA | Capsicum annuum | 100 | 100 99.8 95.9 85.5 | 3 7 15 21 |

* Dat =days after treatment

1. A compound of formula (I) 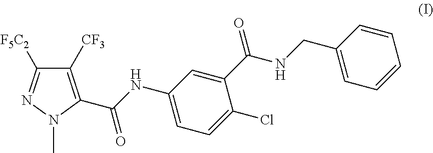
Capable of being used for controlling spider mites from the order of Acari in crops selected from green beans (Phaseolus vulgaris), soya beans (Glycine max), cotton (Gossypium), maize (Zea mays), tea (Camellia sinensis), coffee (Coffea, preferably Coffea arabica and Coffea canephora), grapes (Vitis vinifera, optionally Vitis vinifera L. subsp. vinifera), pome fruit, Solanaceae, stone fruit, optionally almonds (Prunus dulcis), Cucurbitaceae, and nut fruits, under the condition that the the compound of the formula (I) for controlling Tetranychus urticae in green beans (Phaseolus vulgaris) is excluded.

2. Compound according to claim 1, wherein the spider mite is selected from Tetranychus urticae, Tetranychus pacificus, Oligonychus pratensis, Oligonychus coffeae, Polyphagotarsonemus latus and Panonychus ulmi.

3. Compound according to claim 1, wherein the spider mite is a spider mite from the Oligonychus genus.

4. Compound according to claim 3, wherein the spider mite is Oligonychus pratensis.

5. Compound according to claim 4, wherein the crop is maize.

6. Compound according to claim 3, wherein the spider mite is Oligonychus coffeae.

7. Compound according to claim 6, wherein the crop is tea or coffee.

8. Compound according to claim 1, wherein the spider mite is a spider mite from the Tetranychus genus.

9. Compound according to claim 8, in which the spider mite is Tetranychus pacificus.

10. Compound according to claim 9, wherein the crop is almonds or grapes.

11. Compound according to claim 8, in which the spider mite is Tetranychus urticae.

12. Compound according to claim 11, wherein the crop is soya beans, cotton or maize.

13. Compound according to claim 1, wherein the spider mite is Panonychus ulmi.

14. Compound according to claim 1, wherein the spider mite is Polyphagotarsonemus latus.

15. Compound according to claim 13, wherein the crop is a crop selected from the group consisting of green beans (Phaseolus vulgaris), cotton, grapes (Vitis vimfera, optionally Vitis vinifera L. subsp. vinifera), Solanaceae, pome fruit and Cucurbitaceae.
